(19) United States
(12) Patent Application Publication
(10) Pub. No.: US 2012/0114710 A1
(43) Pub. Date: May 10, 2012

(54) CARBON NANOTUBES COMPLEXED WITH MULTIPLE BIOACTIVE AGENTS AND METHODS RELATED THERETO

(76) Inventor: Lynn Kirkpatrick, Houston, TX (US)

(21) Appl. No.: 13/321,098

(22) PCT Filed: May 18, 2010
(86) PCT No.: PCT/US10/35304

§ 371 Date: Jan. 19, 2012

Related U.S. Application Data
(60) Provisional application No. 61/179,162, filed on May 18, 2009.

(51) Int. Cl. A61K 9/00; A61K 48/00; A61K 31/7088; B82Y 5/00

(52) U.S. Cl. 424/400; 514/44 A; 977/742; 977/906

(57) ABSTRACT

The present invention includes fullerene carbon nanotube compositions complexed with multiple bioactive agents and methods related to such fullerene carbon nanotube compositions.

This application claims benefit of priority to U.S. Provisional Patent Application No. 61/179,162 filed on May 18, 2009, which is hereby incorporated by reference in its entirety.

### FIELD

The invention presented herein relates to gene therapy systems. More specifically, the present invention relates to fullerene carbon nanotubes complexed with a plurality of bioactive agents and methods related thereto.

### BACKGROUND

Gene therapy has become an increasingly important mode of treatment for a variety of indications. RNA interference (RNAi), in particular, is a promising treatment method. RNA interference (RNAi) or gene silencing involves reducing the expression of a target gene through mediation by small single- or double-stranded RNAmolecules. These molecules include small interfering RNAs (siRNAs), microRNAs (miRNAs), and small hairpin RNAs (shRNAs), among others.

Numerous gene therapy platforms for the delivery of such molecules are currently available. Within the family of nanotechnology-based gene therapy platforms are carbon nanotubes (CNTs). CNTs can be functionalized to deliver their cargos to cells and organs. However, typically before CNTs can be used in biomedical applications, the hydrophobic nonfunctionalized CNTs must be suspended in aqueous solutions.

### SUMMARY

Embodiments of the present invention provide a CNT composition including a soluble CNT; a first bioactive agent complexed with the CNT, at least a second bioactive agent complexed with the CNT, and a pharmaceutically acceptable carrier. In certain embodiments, the CNT is unagglomerated and nonaggregated.

In other embodiments of the invention, a pharmaceutical composition is provided including a fullerene carbon nanotube, a first siRNA complexed with the fullerene carbon nanotube, at least a second siRNA complexed with the fullerene carbon nanotube, and a pharmaceutically acceptable carrier, wherein the first siRNA is targeted and the second siRNA is untargeted. In various embodiments, the fullerene carbon nanotube is a single-walled carbon nanotube (SWCNT).

In some embodiments, the pharmaceutical composition further comprises at least a third siRNA complexed with the fullerene carbon nanotube.

Further embodiments provide a pharmaceutical composition including a fullerene carbon nanotube, a first siRNA complexed with the fullerene carbon nanotube, at least a second siRNA complexed with the fullerene carbon nanotube, and a pharmaceutically acceptable carrier, wherein the first siRNA is targeted and the second siRNA noncovalently solubilizes the fullerene carbon nanotube into the pharmaceutically acceptable carrier.

Still further embodiments provide a pharmaceutical composition including a fullerene carbon nanotube, a first siRNA complexed with the fullerene carbon nanotube, at least a second siRNA complexed with the fullerene carbon nanotube, and a pharmaceutically acceptable carrier, wherein the first siRNA is targeted to a first target and the second siRNA is targeted to a second target.

The diameter of the fullerene carbon nanotube in one or more embodiments is about 1-5 nm. In other embodiments, the diameter is about 1 nm. The length of the fullerene carbon nanotube in some embodiments is about 500 nm or less. In other embodiments, the length is less than about 400 nm. In yet other embodiments, the length is about 100-300 nm. In still other embodiments, the length is about 125-275 nm. The length in further embodiments is about 150-250 nm. In still other embodiments, the length is about 175-225 nm.

The bioactive agent of the invention may be any bioactive substance known to those of ordinary skill in the art. For example, the bioactive agent of certain embodiments is selected from the group consisting of chemotherapeutic agents, diagnostic agents, prophylactic agents, nutraceutical agents, nucleic acids, proteins, peptides, lipids, carbohydrates, hormones, small molecules, metals, ceramics, drugs, vaccines, immunological agents, and combinations thereof. In one or more preferred embodiments, the bioactive agent includes siRNA. In some aspects of the invention, the bioactive agent includes chemically-modified siRNA. In others, the bioactive agent includes stabilized siRNA.

In certain aspects of the invention, the bioactive agent includes “non-targeting siRNA”, meaning siRNA used for non-sequence-specific effects. A non-limiting example of a non-targeting siRNA is siTox, purchased from Dharmacon Inc. In other aspects, the bioactive agent includes “targeting siRNA” wherein the siRNA is targeted to mRNA. The targeting siRNA may be targeted to any mRNA. In a non-limiting example, the siRNA is targeted to vascular endothelial growth factor (VEGF) mRNA, in which case the sense strand of the siRNA may be AUGUGAAUGCAGACCAAAGAA (SEQ ID NO:1), among others. The siRNA of other embodiments is targeted to endothelial growth factor receptor (EGFR) mRNA, in which case the sense strand may be GUCAGCCUGAACAUAACAU (SEQ ID NO:2) or GUGUAACGGAAUAGGUAUU (SEQ ID NO:3), among others. The siRNA of yet other embodiments is targeted to human epidermal growth factor receptor 2 (HER2) mRNA. In this embodiment, the sense strand of the siRNA may be GGAGCUGGCGGCCUUGUGCCG (SEQ ID NO:4) or UCACAGGGGCCUCCCCAGGAG (SEQ ID NO:5), among others. In other embodiments, the siRNA is targeted to hypoxia-inducible factor 1 alpha (HIF-1a) mRNA, in which case the sense strand of the siRNA may be CCUGUGUCUAAAUCUGAAC (SEQ ID NO:6) or CUACCUUCGUGAUUCUGUUU (SEQ ID NO:7) or GCACAAUAGACAGCGAAAC (SEQ ID NO:8) or CUACUUUCUUAAUGGCUUA (SEQ ID NO:9), among others. In other embodiments, the siRNA is targeted to hypoxia-inducible factor 1 alpha (HIF-1a) mRNA, in which case the sense strand of the siRNA may be 5′ CAAAUACAUGGGAUUAACU[dT][dT]3′ (SEQ. ID. NO:19)) or 5′ GCAACUUGAGGAAGUACCA[dT][dT]3′ (SEQ. ID. NO: 20)).

In further embodiments of the invention, the siRNA is targeted to polo-like kinase 1 (PLK 1), in which case the sense strand may be CAACCAAAGUCGAAUAUUGAUU (SEQ ID NO:10) or CAAGAAGAAUGAAUACAGUUU (SEQ ID NO:11) or GAAGAUGUCCAUGGAAAUAUU (SEQ ID NO:12) or CAACACGCCUCAUCCUCUAUU (SEQ ID NO:13), among others. The siRNA of yet other embodiments is targeted to Kinesin superfamily protein (Kif11), in which case the sense strand may be CGUCUUUAGAUUCCUAUAU (SEQ ID NO:14) or GUUGUUCCUACUUCAGAUA (SEQ ID NO:15) or GUCGUCUUUAGAUUCCUAU (SEQ ID NO:16) or GAUCUACCGAAAGAGUCAU-3′ (SEQ ID NO:17), among others.

In certain aspects of the present invention, the fullerene carbon nanotube complexes may be optimized with a specific ratio of complexed to noncomplexed surface area, such that the fullerene carbon nanotubes are solubilized into solution and a therapeutically effective amount of a the first and/or at least a second bioactive agent is delivered. Any amount of surface area of the fullerene carbon nanotube may be complexed with the bioactive agents. For example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or about 100% of the surface area of the fullerene carbon nanotube may be complexed with one or more bioactive agents, or any range of surface areas derivable therein may be complexed with one or more bioactive agents.

The pharmaceutically acceptable carrier of certain embodiments is liquid. In some aspects of the invention, the pharmaceutically acceptable carrier is water. In other aspects, the pharmaceutically acceptable carrier is an isotonic salt solution and in other aspects, an isotonic sugar solution. The pharmaceutically acceptable carrier of further aspects is aqueous polyethylene glycol (PEG) solution. In yet others, an organic solvent dissolved in isotonic aqueous solution. In still other aspects, the pharmaceutically acceptable carrier is an aqueous buffer solution.

Embodiments hereof provide a fullerene carbon nanotube composition including a fullerene carbon nanotube, a first bioactive agent complexed with the fullerene carbon nanotube, at least a second bioactive agent complexed with the fullerene carbon nanotube, and a pharmaceutically acceptable carrier wherein the fullerene carbon nanotube composition is internalized in treated cells in media containing serum at a rate measured in vitro that substantially corresponds to the following: (i) from about 0.01 to about 30% of the total amount of treated cells internalize the fullerene carbon nanotube composition after about 1 hour of measurement; (ii) from about 20 to about 90% of the total amount of treated cells internalize the fullerene carbon nanotube composition after about 3 hours of measurement; and (iii) not less than about 95% of the total amount of treated cells internalize the fullerene carbon nanotube composition after about 24 hours of measurement. In some embodiments, one or more of the bioactive agents dissociates from the fullerene carbon nanotube when internalized in the treated cell. In other embodiments, one or more of the bioactive agents remains complexed with the fullerene carbon nanotube when internalized in the treated cell.

The pharmaceutical composition in one or more embodiments of the invention provides delivery of an effective amount of multiple siRNA. Delivery of the effective amount of multiple siRNA reduces the expression of a target nucleic acid when compared to multiple strands of siRNA not complexed to the fullerene carbon nanotube.

Embodiments hereof provide a method of reducing the expression of a targeted gene in cell culture, including delivering an effective amount of a fullerene carbon nanotube composition comprising a fullerene carbon nanotube, a first siRNA complexed with the fullerene carbon nanotube, at least a second siRNA complexed with the fullerene carbon nanotube, and a pharmaceutically acceptable carrier, wherein the first siRNA is targeted and the second siRNA is untargeted.

Other embodiments are directed to a method of reducing the expression of a targeted gene in cell culture, including delivering an effective amount of a fullerene carbon nanotube composition comprising a fullerene carbon nanotube, a first siRNA complexed with the fullerene carbon nanotube, at least a second siRNA complexed with the fullerene carbon nanotube, and a pharmaceutically acceptable carrier, wherein the first siRNA is targeted and the second siRNA noncovalently solubilizes the fullerene carbon nanotube into the pharmaceutically acceptable carrier.

Still further embodiments of the invention are directed to a method of reducing the expression of a targeted gene in cell culture, including delivering an effective amount of a fullerene carbon nanotube composition comprising a fullerene carbon nanotube, a first siRNA complexed with the fullerene carbon nanotube, at least a second siRNA complexed with the fullerene carbon nanotube, and a pharmaceutically acceptable carrier, wherein the first siRNA is targeted to a first target and the second siRNA is targeted to a second target.

In other embodiments, a method of effectively silencing a targeted gene in vivo is provided, including administering to a subject an effective amount of a fullerene carbon nanotube composition comprising a fullerene carbon nanotube, a first siRNA complexed with the fullerene carbon nanotube, at least a second siRNA complexed with the fullerene carbon nanotube, and a pharmaceutically acceptable carrier, wherein the first siRNA is targeted and the second siRNA is untargeted.

Methods of effectively silencing a targeted gene in vivo of yet other embodiments includes administering to a subject an effective amount of a fullerene carbon nanotube composition comprising a fullerene carbon nanotube, a first siRNA complexed with the fullerene carbon nanotube, at least a second siRNA complexed with the fullerene carbon nanotube, and a pharmaceutically acceptable carrier, wherein the first siRNA is targeted and the second siRNA noncovalently solubilizes the fullerene carbon nanotube into the pharmaceutically acceptable carrier.

In still other embodiments of the invention, a method of effectively silencing a targeted gene in vivo is provided, including administering to a subject an effective amount of a fullerene carbon nanotube composition comprising a fullerene carbon nanotube, a first siRNA complexed with the fullerene carbon nanotube, at least a second siRNA complexed with the fullerene carbon nanotube, and a pharmaceutically acceptable carrier, wherein the first siRNA is targeted to a first target and the second siRNA is targeted to a second target.

### BRIEF DESCRIPTION OF THE DRAWINGS

FIG. 1 depicts a Western blot analysis of some embodiments of the invention.

FIG. 2 is a graph comparing the emission fluorescence spectrum of SWCNT solutions of siEGFR single payload (E+SW), siTRX single payload (T+SW) and siEGFR/siTRX SWCNT double payload in accordance with some embodiments of the invention.

### DESCRIPTION

This invention is not limited to the particular compositions, sizes or methodologies described, as these may vary. It is specifically contemplated that any limitation discussed with respect to one embodiment of the invention may or may not apply to any other embodiment of the invention. Furthermore, any composition of the invention may be used in any method of the invention, and any method of the invention may be used to produce or to utilize any composition of the invention.

In addition, the terminology used in the description describes particular versions or embodiments only and is not intended to limit the scope of the present invention. Unless defined otherwise, all technical and scientific terms used herein have the same meanings as commonly understood by one of ordinary skill in the art. In case of conflict, the patent specification, including definitions, will prevail.

As used herein, the singular forms “a”, “an” and “the” include plural reference unless the context clearly dictates otherwise.

As used herein, the term “about” means plus or minus 10% of the numerical value of the number with which it is being used. Therefore, about 50% means in the range of 45% - 55%.

The term “agglomeration”, as used herein, refers to the formation of a cohesive mass consisting of carbon nanotube subunits held together by relatively weak forces (for example, van der Waals or capillary forces) that may break apart into subunits upon processing, for example. The resulting structure is called an “agglomerate.”

As used herein, the term “aggregation” refers to the formation of a discrete group of carbon nanotubes in which the various individual components are not easily broken apart, such as in the case of nanotube bundles that are strongly bonded together. The resulting structure is called an “aggregate.”

As used herein, the term “bioactive agent” means a compound utilized to image, impact, treat, combat, ameliorate, prevent or improve an unwanted condition or disease of a patient. The bioactive agent may modulate any number of biological functions in

[symbol]

The term “carbon nanotube” (CNT), as used herein, refers to a structural constituent, which may take any of the forms described herein and other forms known in the art. In preferred embodiments, the carbon nanotube is a fifflerene nanotube. In various embodiments, the fullerene carbon nanotube is a single-walled carbon nanotube (SWCNT). Fullerene carbon nanotubes are generally made of a single, continuous sheet of hexagonal graphene joined to form a tube with virtually no defects, typically with a hemifullerene cap at either end. Fullerene carbon nanotubes have a molecular structure substantially the same as buckminsterfullerene, but in a cylindrical form. The term carbon nanotube may refer to either a fullerene carbon nanotube with hemispherical caps attached, or it may refer to one derived from such a closed tube by cutting, etching off the ends, or other means. Alternatively, fullerene carbon nanotubes may be constructed of some number of single-walled fullerene nanotubes arranged one inside another, sometimes referred to as multi-walled carbon nanotubes (MWCNTs). The term carbon nanotube, as used herein, may further include structures that are not entirely carbon, such as metals, small-gap semiconductors or large-gap semiconductors. For example, boron carbon nitride (BCN) nanotubes are included in the definition of carbon nanotube.

A “disease” or “health-related condition”, as used herein, can be any pathological condition of a body part, an organ, or a system resulting from any cause, such as infection, genetic defect, and/or environmental stress. The cause may or may not be known. The present invention may be used to treat or prevent any disease or health-related condition in a subject. Examples of such diseases may include, for example, infectious diseases, inflammatory diseases, hyperproliferative diseases such as cancer, degenerative diseases, and so forth. For example, fullerene carbon nanotube complexes of the invention may be administered to treat cancer. The cancer may be a solid tumor, metastatic cancer, or non-metastatic cancer. In certain embodiments, the cancer may originate in the bladder, blood, bone, bone marrow, brain, breast, colon, esophagus, duodenum, small intestine, large intestine, colon, rectum, anus, gum, head, kidney, liver, lung, nasopharynx, neck, ovary, prostate, skin, stomach, testis, tongue, or uterus.

The term “diseased tissue”, as used herein, refers to tissue or cells associated with solid tumor cancers of any type, such as bone, lung, vascular, neuronal, colon, ovarian, breast and prostate cancer. The term diseased tissue may also refer to tissue or cells of the immune system, such as tissue or cells effected by AIDS; pathogen-borne diseases, which can be bacterial, viral, parasitic, or fungal, examples of pathogen-borne diseases include HIV, tuberculosis and malaria; hormone-related diseases, such as obesity; vascular system diseases such as macular degeneration; central nervous system diseases, such as multiple sclerosis; and undesirable matter, such as adverse angiogenesis, restenosis amyloidosis, toxins, reaction-by-products associated with organ transplants, and other abnormal cell or tissue growth.

An “effective amount” or “therapeutically effective amount” of a composition, as used herein, refers to an amount of a biologically active molecule or complex or derivative thereof sufficient to exhibit a detectable therapeutic effect without undue adverse side effects (such as toxicity, irritation and allergic response) commensurate with a reasonable benefit/risk ratio when used in the manner of the invention. The therapeutic effect may include, for example but not by way of limitation, inhibiting the growth of undesired tissue or malignant cells. The effective amount for a subject will depend upon the type of subject, the subject's size and health, the nature and severity of the condition to be treated, the method of administration, the duration of treatment, the nature of concurrent therapy (if any), the specific formulations employed, and the like.

“Gene silencing” refers to the suppression of gene expression, e.g., transgene, heterologous gene and/or endogenous gene expression. Gene silencing may be mediated through processes that affect transcription and/or through processes that affect post-transcriptional mechanisms. In some embodiments, gene silencing occurs when siRNA initiates the degradation of the mRNA of a gene of interest in a sequence-specific manner via RNA interference.

The terms “include”, “comprise” and “have” and their conjugates, as used herein, mean “including but not necessarily limited to.”

“Knock-down” or “knock-down technology” refers to a technique of gene silencing in which the expression of a target gene is reduced as compared to the gene expression prior to the introduction of the siRNA, which can lead to the inhibition of production of the target gene product.

As used herein, the terms “nonaggregated”, “unagglomeration” and “unagglomerated” refer to a state of dispersion in a suspension.

The term “nucleic acid” refers to deoxyribonucleotides or ribonucleotides and polymers thereof in either single- or double-stranded form, composed of monomers (nucleotides) containing a sugar, phosphate and a base that is either a purine or pyrimidine. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues.

The term “patient”, as used herein, includes human and veterinary subjects.

As used herein, a “pharmaceutically acceptable carrier” includes any and all pharmaceutically acceptable solvents, suspending agents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, vehicle, such like materials and combinations thereof, for delivering the complexes of the present invention to the patient, as would be known to one of ordinary skill in the art.

“RNA interference (RNAi)” is the process of sequence-specific, posttranscriptional gene silencing initiated by siRNA. RNAi is seen in a number of organisms such as Drosophila, nematodes, fungi and plants, and is believed to be involved in anti-viral defense, modulation of transposon activity, and regulation of gene expression. During RNAi, siRNA induces degradation of target mRNA with consequent sequence-specific inhibition of gene expression.

The terms “small interfering” or “short interfering RNA” or “siRNA” refer a RNA duplex of nucleotides that is targeted to a gene of interest. A “RNA duplex” refers to the structure formed by the complementary pairing between two regions of a RNA molecule. siRNA is “targeted” to a gene in that the nucleotide sequence of the duplex portion of the siRNA is complementary to a nucleotide sequence of the targeted gene. In some embodiments, the length of the duplex of siRNA is less than 30 nucleotides. In some embodiments, the duplex can be 29, 28, 27, 26, 25, 24, 23, 22, 21, 20, 19, 18, 17, 16, 15, 14, 13, 12, 11 or 10 nucleotides in length. In some embodiments, the length of the duplex is 19-25 nucleotides in length. The RNA duplex portion of the siRNA can be part of a hairpin structure. In addition to the duplex portion, the hairpin structure may contain a loop portion positioned between the two sequences that form the duplex. The loop can vary in length. In some embodiments the loop is 5, 6, 7, 8, 9, 10, 11, 12 or 13 nucleotides in length. The hairpin structure can also contain 3′ or 5′ overhang portions. In some embodiments, the overhang is a 3′ or a 5′ overhang 0, 1, 2, 3, 4 or 5 nucleotides in length. In some embodiments, siRNA refers to a class of double-stranded RNA molecules including, for example, chemically-modified siRNA, stabilized siRNA, targeting siRNA, and non-targeting siRNA.

The term “stable” or “stabilized” means a solution or suspension in a fluid phase wherein solid components (i.e., nanotubes and bioactive agents) possess stability against aggregation and agglomeration sufficient to allow manufacture and delivery to a cell and which maintain the integrity of the compound for a sufficient period of time to be detected and preferably for a sufficient period of time to be useful for the purposes detailed herein.

A “subject”, as used herein, refers to either a human or non-human, such as primates, mammals, and vertebrates. In particular embodiments, the subject is a human.

“Treatment” and “treating” refer to administration or application of a pharmaceutical composition embodied in the invention to a subject or performance of a procedure or modality on a subject for the purpose of obtaining a therapeutic benefit of a disease or health-related condition. For example, a treatment may include administration of a therapeutically effective amount of a pharmaceutical composition that inhibits the expression of a gene for the purposes of minimizing the growth or invasion of a tumor, such as a colorectal cancer.

Embodiments of the present invention provide a fullerene carbon nanotube composition for delivery of multiple bioactive agents including a fullerene carbon nanotube, a first bioactive agent complexed with the fullerene carbon nanotube, and at least a second bioactive agent complexed with the fullerene carbon nanotube. In some embodiments, one or more of the bioactive agents may disperse the fullerene carbon nanotubes.

The diameter of the fullerene carbon nanotube in one or more embodiments is about 1-5 nm. In certain embodiments, the diameter is about 1 nm. The length of the fullerene carbon nanotube in some embodiments is about 500 nm or less. In other embodiments, the length is less than about 400 nm. In yet other embodiments, the length is about 100-300 nm. In still other embodiments, the length is about 125-275 nm. The length in further embodiments is about 150-250 nm. In still other embodiments, the length is about 175-225 nm.

In certain aspects of the present invention, the fullerene carbon nanotube complexes may be optimized with a specific ratio of complexed to noncomplexed surface area, such that the fullerene carbon nanotubes are solubilized into solution and a therapeutically effective amount of one or more bioactive agents is delivered. Any amount of surface area of the fullerene carbon nanotube may be complexed with one or more bioactive agents. For example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or about 100% of the surface area of the fullerene carbon nanotube may be complexed with one or more bioactive agents, or any range of surface areas derivable therein may be complexed with one or more bioactive agents.

The bioactive agent of embodiments of the invention may include any bioactive agent known to those of ordinary skill in the art. For example, may be selected from the group consisting of chemotherapeutic agents such as, for example, doxorubicin, diagnostic agents, prophylactic agents, nutraceutical agents, nucleic acids, proteins, peptides, lipids, carbohydrates, hormones, small molecules, metals, ceramics, drugs, vaccines, immunological agents, and combinations thereof. In one or more preferred embodiments, the bioactive agent comprises siRNA and numerous siRNA sequences can be utilized to complex the fullerene carbon nanotubes of the invention. Further, in some aspects of the invention, a siRNA may solubilize the fullerene carbon nanotubes. The bioactive agent of certain aspects of the invention comprises chemically-modified siRNA. In others, the bioactive agent includes stabilized siRNA.

In embodiments of the invention, the fullerene carbon nanotubes can be complexed with any number of bioactive agents. In addition, the fullerene carbon nanotubes may be optionally complexed with one or more substances that are not bioactive. In certain embodiments, the fullerene carbon nanotube may be optionally complexed with a composition that includes one or more bioactive agents and one or more non-bioactive substances.

In certain aspects of the invention, the bioactive agent includes “non-targeting siRNA”, meaning siRNA used for non-sequence-specific effects. A non-limiting example of a non-targeting siRNA is siTox, purchased from Dharmacon Inc. In other aspects, the bioactive agent includes “targeting siRNA” wherein the siRNA is targeted to

[symbol]

[symbol]

In some embodiments, the siRNA may have one or more dT overhangs. In some embodiments, the siRNA may have a dTdT overhang. In some embodiments, the siRNA may have from 1 to 8 overhangs. In other embodiments, the siRNA may have no dT overhangs. In particular, in some embodiments, the specific sequences disclosed may be used with or with the dT overhang.

In a non-limiting example, the siRNA is targeted to vascular endothelial growth factor (VEGF) mRNA, in which case the sense strand of the siRNA comprises AUGUGAAUGCAGACCAAAGAA (SEQ ID NO:1), among others. The.siRNA of other embodiments is targeted to endothelial growth factor receptor (EGFR) mRNA, in which case the sense strand comprises GUCAGCCUGAACAUAACAU (SEQ ID NO:2) or GUGUAACGGAAUAGGUAUU (SEQ ID NO:3), among others. The siRNA of yet other embodiments is targeted to human epidermal growth factor receptor 2 (HER2) mRNA. In this embodiment, the sense strand of the siRNA comprises GGAGCUGGCGGCCUUGUGCCG (SEQ ID NO:4) or UCACAGGGGCCUCCCCAGGAG (SEQ ID NO:5), among others. In other embodiments, the siRNA is targeted to hypoxia-inducible factor 1 alpha (HIF-1a) mRNA, in which case the sense strand of the siRNA comprises CCUGUGUCUAAAUCUGAAC (SEQ ID NO:6) or CUACCUUCGUGAUUCUGUUU (SEQ ID NO:7) or GCACAAUAGACAGCGAAAC (SEQ ID NO:8) or CUACUUUCUUAAUGGCUUA (SEQ ID NO:9), among others. In other embodiments, the siRNA is targeted to hypoxia-inducible factor 1 alpha (HIF-1a) mRNA, in which case the sense strand of the siRNA comprises 5′ CAAAUACAUGGGAUUAACU[dT][dT]3′ (SEQ. ID. NO:19)) or 5′ GCAACUUGAGGAAGUACCA[dT][dT]3′ (SEQ. ID. NO: 20)). In further embodiments of the invention, the siRNA is targeted to polo-like kinase 1 (PLK1), in which case the sense strand comprises CAACCAAAGUCGAAUAUUGAUU (SEQ ID NO:10) or CAAGAAGAAUGAAUACAGUUU (SEQ ID NO:11) or GAAGAUGUCCAUGGAAAUAUU (SEQ ID NO:12) or CAACACGCCUCAUCCUCUAUU (SEQ ID NO:13), among others. The siRNA of yet other embodiments is targeted to Kinesin superfamily protein (Kif11), in which case the sense strand comprises CGUCUUUAGAUUCCUAUAU (SEQ ID NO:14) or GUUGUUCCUACUUCAGAUA (SEQ ID NO:15) or GUCGUCUUUAGAUUCCUAU (SEQ ID NO:16) or GAUCUACCGAAAGAGUCAU-3′ (SEQ ID NO:17), among others. In further embodiments of the invention, the siRNA is targeted to Thioredoxin (TRX) in which case the sense strand comprises 5′ CCAGUUGCCAUCUGCGUGA[dT][dT] 3′ (SEQ. ID NO: 21), 5′ CUUGGACGCUGCAGGUGAU[dT][dT] 3′ (SEQ.ID.NO:22), 5′ AUUCCAACGUGAUAUUCCU[dT][dT] 3′ (SEQ.ID.NO:23), or 5′ GCCAUCUGCGUGACAAUAA[dT][dT] 3′ (SEQ.ID.NO:24), among others. In further embodiments of the invention, the siRNA is targeed to Epidermal growth factor receptor (EGFR). In such cases, the sense strand comprises 5′ CUAUGUGCAGAGGAAUUAU[dT][dT] 3′ (SEQ. ID. NO:25), 5′ GAUCUUUCCUUCUUAAAGA[dT][dT] 3′ (SEQ. ID. NO:26), 5′ GAGGAAAUAUGUACUACGA[dT][dT] 3′ (SEQ. ID. NO:27), 5′ GACAUAGUCAGCAGUGACU[dT][dT] 3′ (SEQ. ID. NO:28) among others. In further embodiments of the invention, the siRNA is targeted to v-Ki-ras2 Kirsten rat sarcoma 2 viral oncogene homolog (KRAS) in which case the sense strand comprises 5′ GUGCAAUGAGGGACCAGUA[dT][dT] 3′ (SEQ. ID. NO:29), or 5′ GUCUCUUGGAUAUUCUCGA[dT][dT] 3′ (SEQ. ID. NO:30), among others.

In a non-limiting example, the siRNA is targeted to vascular endothelial growth factor (VEGF) mRNA, in which case the sense strand of the siRNA consists of AUGUGAAUGCAGACCAAAGAA (SEQ ID NO:1), among others. The siRNA of other embodiments is targeted to endothelial growth factor receptor (EGFR) mRNA, in which case the sense strand consists of GUCAGCCUGAACAUAACAU (SEQ ID NO:2) or GUGUAACGGAAUAGGUAUU (SEQ ID NO:3), among others. The siRNA of yet other embodiments is targeted to human epidermal growth factor receptor 2 (HER2) mRNA. In this embodiment, the sense strand of the siRNA consists of GGAGCUGGCGGCCUUGUGCCG (SEQ ID NO:4) or UCACAGGGGCCUCCCCAGGAG (SEQ ID NO:5), among others. In other embodiments, the siRNA is targeted to hypoxia-inducible factor 1 alpha (HIF-1a) mRNA, in which case the sense strand of the siRNA consists of CCUGUGUCUAAAUCUGAAC (SEQ ID NO:6) or CUACCUUCGUGAUUCUGUUU (SEQ ID NO:7) or GCACAAUAGACAGCGAAAC (SEQ ID NO:8) or CUACUUUCUUAAUGGCUUA (SEQ ID NO:9), among others. In other embodiments, the siRNA is targeted to hypoxia-inducible factor 1 alpha (HIF-1a) mRNA, in which case the sense strand of the siRNA consists of 5′ CAAAUACAUGGGAUUAACU[dT][dT]3′ (SEQ. ID. NO:19)) or 5′ GCAACUUGAGGAAGUACCA[dT][dT]3′ (SEQ. ID. NO: 20)). In further embodiments of the invention, the siRNA is targeted to polo-like kinase 1 (PLK1), in which case the sense strand consists of CAACCAAAGUCGAAUAUUGAUU (SEQ ID NO:10) or CAAGAAGAAUGAAUACAGUUU (SEQ ID NO:11) or GAAGAUGUCCAUGGAAAUAUU (SEQ ID NO:12) or CAACACGCCUCAUCCUCUAUU (SEQ ID NO:13), among others. The siRNA of yet other embodiments is targeted to Kinesin superfamily protein (Kif11), in which case the sense strand consists of CGUCUUUAGAUUCCUAUAU (SEQ ID NO:14) or GUUGUUCCUACUUCAGAUA (SEQ ID NO:15) or GUCGUCUUUAGAUUCCUAU (SEQ ID NO:16) or GAUCUACCGAAAGAGUCAU-3′ (SEQ ID NO:17), among others. In further embodiments of the invention, the siRNA is targeted to Thioredoxin (TRX) in which case the sense strand consists of 5′ CCAGUUGCCAUCUGCGUGA[dT][dT] 3′ (SEQ. ID NO: 21), 5′ CUUGGACGCUGCAGGUGAU[dT][dT] 3′ (SEQ.ID.NO:22), 5′ AUUCCAACGUGAUAUUCCU[dT][dT] 3′ (SEQ.ID.NO:23), or 5′ GCCAUCUGCGUGACAAUAA[dT][dT] 3′ (SEQ.ID.NO:24), among others. In further embodiments of the invention, the siRNA is targeed to Epidermal growth factor receptor (EGFR). In such cases, the sense strand consists of 5′ CUAUGUGCAGAGGAAUUAU[dT][dT] 3′ (SEQ. ID. NO:25), 5′ GAUCUUUCCUUCUUAAAGA[dT][dT] 3′ (SEQ. ID. NO:26), 5′ GAGGAAAUAUGUACUACGA[dT][dT] 3′ (SEQ. ID. NO:27), 5′ GACAUAGUCAGCAGUGACU[dT][dT] 3′ (SEQ. ID. NO:28) among others. In further embodiments of the invention, the siRNA is targeted to v-Ki-ras2 Kirsten rat sarcoma 2 viral oncogene homolog (KRAS) in which case the sense strand consists of 5′ GUGCAAUGAGGGACCAGUA[dT][dT] 3′ (SEQ. ID. NO:29), or 5′ GUCUCUUGGAUAUUCUCGA[dT][dT] 3′ (SEQ. ID. NO:30), among others.

Any suitable siRNA may be used for any of the first siRNA, second siRNA, third siRNA or any additional number of siRNAs employed in various embodiments of the invention. Particularly, and of the specific targets described herein and the sequences disclosed herein can be used in any and all of the first siRNA, second siRNA, third siRNA or any additional number of siRNAs complexed to the nanotubes.

In some embodiments, the fullerene carbon nanotubes of the present invention may be coupled or functionalized with a “functional group”, wherein such functional group links one or more of the bioactive agents to the fullerene carbon nanotube. The functional group of embodiments of the invention may be any linker group known to those of ordinary skill in the art, such as, for example, carboxyl groups, carbonyl groups, hydroxyl groups, butylated hydroxytoluene (BHT), and polyethylene glycol (PEG). In certain aspects of the invention, the functional group may include one or more of the bioactive agents themselves. In some embodiments, one or more of the bioactive agents are covalently bound to the fullerene carbon nanotube. In still other embodiments, one or more of the bioactive agents are noncovalently bound to the fullerene carbon nanotubes. In one or more preferred embodiments, the bioactive agent comprises siRNA and numerous siRNA sequences can be utilized to complex the fullerene carbon nanotubes of the invention. Further, in some aspects of the invention, one or more siRNA may solubilize the fullerene carbon nanotubes.

The fullerene carbon nanotube complexes may be combined with an acceptable carrier to produce a pharmaceutical formulation, according to another aspect of the invention. In various embodiments of the invention, a pharmaceutical composition is provided including a fullerene carbon nanotube, a first siRNA complexed with the fullerene carbon nanotube, at least a second siRNA complexed with the fullerene carbon nanotube, and a pharmaceutically acceptable carrier, wherein the first siRNA is targeted and the second siRNA is untargeted.

Further embodiments provide a pharmaceutical composition including a fullerene carbon nanotube, a first siRNA complexed with the fullerene carbon nanotube, at least a second siRNA complexed with the fullerene carbon nanotube, and a pharmaceutically acceptable carrier, wherein the first siRNA is targeted and the second siRNA noncovalently solubilizes the fullerene carbon nanotube into the pharmaceutically acceptable carrier.

Still other embodiments provide a pharmaceutical composition including a fullerene carbon nanotube, a first siRNA complexed with the fullerene carbon nanotube, at least a second siRNA complexed with the fullerene carbon nanotube, and a pharmaceutically acceptable carrier, wherein the first siRNA is targeted to a first target and the second siRNA is targeted to a second target.

The pharmaceutically acceptable carrier of embodiments of the invention may be any carrier known to those of ordinary skill in the art. The carrier may be liquid or solid and is selected with the planned manner of administration in mind. Examples of

[symbol]

As would be appreciated by one of skill in this art, the pharmaceutically acceptable carrier may be selected based on factors including, but not limited to, route of administration, location of the disease tissue, the number and type of bioactive agent(s) being delivered, and/or time course of delivery of the bioactive agent(s). For example, where clinical application of the carbon nanotube (CNT) complexes of the present invention is undertaken, it will generally be beneficial to prepare the CNT complexes as a pharmaceutical composition appropriate for the intended application. This will typically entail preparing a pharmaceutical composition that is essentially free of pyrogens, as well as any other impurities that could be harmful to humans or animals. In preparing a pharmaceutical composition, one may also employ appropriate buffers to render the complex stable and allow for uptake by target cells.

The pharmaceutically acceptable carrier embodied in the invention is preferably formulated for administration to a human, although in certain embodiments it may be desirable to use a pharmaceutically acceptable carrier that is formulated for administration to a non-human animal, but which would not be acceptable (e.g., due to governmental regulations) for administration to a human. Except insofar as any conventional carrier is incompatible with the bioactive agents, its use in the therapeutic or pharmaceutical compositions is contemplated.

The pharmaceutically acceptable carrier of certain embodiments is liquid. In some aspects of the invention, the pharmaceutically acceptable carrier is water. In other aspects, the pharmaceutically acceptable carrier is an isotonic salt solution and in other aspects, an isotonic sugar solution. The pharmaceutically acceptable carrier of further aspects is aqueous polyethylene glycol (PEG) solution. In yet others, an organic solvent dissolved in isotonic aqueous solution. In still other aspects, the pharmaceutically acceptable carrier is an aqueous buffer solution. The pH and exact concentration of the various components the pharmaceutical composition are adjusted according to well-known parameters.

The pharmaceutical composition in one or more embodiments of the invention provides delivery of an effective amount of multiple bioactive agents, such as, for example multiple siRNA or one or more siRNA in combination with other bioactive agents such as, for example, chemotherapeutic agents such as, for example, doxorubicin, diagnostic agents, prophylactic agents, neutraceutical agents, nucleic acids, proteins, peptides, lipids, carbohydrates, hormones, small molecules, metals, ceramics, drugs, vaccines, immunological agents, and combinations thereof. Delivery of the effective amount of the pharmaceutical composition reduces the expression of a target nucleic acid when compared one or more siRNA not complexed to the fullerene carbon nanotube.

The CNT complexes embodied herein can be used for a variety of applications, such as, without limitation, drug delivery, gene therapy, medical diagnosis and for medical therapeutics for cancer, pathogen-borne diseases, hormone-related diseases, reaction-by-products associated with organ transplants, and other abnormal cell or tissue growth.

Embodiments hereof provide a CNT composition including a CNT, a first bioactive agent complexed with the CNT, at least a second bioactive agent complexed with the CNT, and a pharmaceutically acceptable carrier wherein the CNT composition is internalized in treated cells in media containing serum at a rate measured in vitro that substantially corresponds to the following: (i) from about 0.01 to about 30% of the total amount of treated cells internalize the CNT composition after about 1 hour of measurement; (ii) from about 20 to about 90% of the total amount of treated cells internalize the CNT composition after about 3 hours of measurement; and (iii) not less than about 95% of the total amount of treated cells internalize the CNT composition after about 24 hours of measurement. In some embodiments, the first and/or the second bioactive agent dissociates from the CNT when internalized in the treated cell. In other embodiments, the the first and/or the second bioactive agent remains complexed with the CNT when internalized in the treated cell.

In other embodiments, a method of reducing the expression of a targeted gene in cell culture is provided, including delivering an effective amount of a CNT composition comprising a CNT, a first siRNA complexed with the CNT, at least a second siRNA complexed with the CNT, and a pharmaceutically acceptable carrier, wherein the first siRNA is targeted and the second siRNA is untargeted.

Other embodiments are directed to a method of reducing the expression of a targeted gene in cell culture, including delivering an effective amount of a CNT composition comprising a CNT, a first siRNA complexed with the CNT, at least a second siRNA complexed with the CNT, and a pharmaceutically acceptable carrier, wherein the first siRNA is targeted and the second siRNA noncovalently solubilizes the CNT into the pharmaceutically acceptable carrier.

Still further embodiments of the invention are directed to a method of reducing the expression of a targeted gene in cell culture, including delivering an effective amount of a CNT composition comprising a CNT, a first siRNA complexed with the CNT, at least a second siRNA complexed with the CNT, and a pharmaceutically acceptable carrier, wherein the first siRNA is targeted to a first target and the second siRNA is targeted to a second target.

In other embodiments, a method of effectively silencing a targeted gene in vivo is provided, including administering to a subject an effective amount of a CNT composition comprising a CNT, a first siRNA complexed with the CNT, at least a second siRNA complexed with the CNT, and a pharmaceutically acceptable carrier, wherein the first siRNA is targeted and the second siRNA is untargeted.

Methods of effectively silencing a targeted gene in vivo of other embodiments includes administering to a subject an effective amount of a CNT composition comprising a CNT, a first siRNA complexed with the CNT, at least a second siRNA complexed with the CNT, and a pharmaceutically acceptable carrier, wherein the first siRNA is targeted and the second siRNA noncovalently solubilizes the CNT into the pharmaceutically acceptable carrier.

In still other embodiments of the invention, a method of effectively silencing a targeted gene in vivo is provided, including administering to a subject an effective amount of a CNT composition comprising a CNT, a first siRNA complexed with the CNT, at least a second siRNA complexed with the CNT, and a pharmaceutically acceptable carrier, wherein the first siRNA is targeted to a first target and the second siRNA is targeted to a second target.

One aspect of the invention includes methods for treating a disease using CNT compositions. The diseases that may be treated using methods of the present invention encompass a broad range of indications, as CNT complexes of embodiments of the present invention have the potential to function as a serum-insensitive, wide range transfection agent to deliver siRNA into cells to induce a response. In other aspects of the present invention, CNT complexes may be used to silence target genes with a high degree of specificity. The CNT complexes can be used for a variety of applications, such as, without limitation, drug delivery, gene therapy, medical diagnosis and for medical therapeutics for cancer, pathogen-borne diseases, hormone-related diseases, reaction-by-products associated with organ transplants, and other abnormal cell or tissue growth.

In some embodiments of the invention, the methods include identifying a patient in need of treatment. A patient may be identified, for example, based on taking a patient history, based on findings during clinical examination, based on health screenings, or by patient self-referral.

Various routes of administration are contemplated in aspects of the invention. In particular embodiments, the CNT complexes are administered to a subject systemically. In other embodiments, methods of administration may include, but are not limited to, intravascular injection, intravenous injection, intraperitoneal injection, subcutaneous injection, intramuscular injection, transmucosal administration, oral administration, topical administration, local administration, or regional administration. In some embodiments, the CNT complexes are administered intraoperatively. In other embodiments, the CNT complexes are administered via a drug delivery device. According to other embodiments of the invention, the CNT complexes necessitate only a single or very few treatment sessions to provide therapeutic treatment, which ultimately may facilitate patient compliance.

In particular embodiments, oral formulations of the CNT complexes include such typical excipients as, for example, pharmaceutical grades of mannitol, lactose, starch, magnesium stearate, sodium saccharine, cellulose, magnesium carbonate and the like. Topical administration may be particularly advantageous for the treatment of skin cancers, to prevent chemotherapy-induced alopecia or other dermal hyperproliferative disorder. Such compositions would normally be administered as pharmaceutically acceptable compositions that include physiologically acceptable carriers, buffers or other excipients. For treatment of conditions of the lungs, or respiratory tract, aerosol delivery can be used. In such a case, volume of the aerosol may be between about 0.01 ml and 0.5 ml.

The amount of CNT complexes administered to a patient may vary. The term “unit dose” or “dosage” refers to physically discrete units suitable for use in a subject, each unit containing a predetermined-quantity of the therapeutic composition calculated to produce the desired responses discussed above in association with its administration, i.e., the appropriate route and treatment regimen. The quantity to be administered, both according to number of treatments and unit dose, depends on the protection or effect desired. Precise amounts of the pharmaceutical compositions also depend on the judgment of the practitioner and are peculiar to each individual. Factors affecting the dose include the physical and clinical state of the patient, the intended goal of treatment (e.g., alleviation of symptoms versus cure) and the potency, stability and toxicity of the particular therapeutic substance. The amount of CNT complexes administered to a patient may vary and may depend on the size, age, and health of the patient, the number and types of bioactive agents to be delivered, the indication being treated, and the location of diseased tissue. Moreover, the dosage may vary depending on the mode of administration.

In various aspects of the invention, a kit is envisioned containing CNT complexes as set forth herein. In some embodiments, the present invention contemplates a kit for preparing and/or administering such CNT complexes. The kit may comprise one or more sealed vials containing any of the CNT complexes or reagents for preparing any of such CNT complexes. In certain embodiments, the kit may also comprise a “suitable container means”, which is a container that will not react with components of the kit, such as, for example, an eppendorf tube, an assay plate, a syringe, a bottle, or a tube. Such suitable container means may be made from sterilizable materials such as plastic or glass.

The kit may further include an instruction sheet that outlines the procedural steps of the methods, and will follow substantially the same procedures as described herein or are known to those of ordinary skill. The instruction information may be in a computer readable media containing machine-readable instructions that, when executed using a computer, cause the display of a real or virtual procedure of delivering and/or administering a therapeutically effective amount of the CNT complexes of the present invention.

### EXAMPLES

In order that the invention disclosed herein may be more efficiently understood, examples are provided. The following examples are for illustrative purposes only and are not to be construed as limiting the invention in any manner.

### Example 1.1.

### Preparation of Noncovalent Complexes of SWCNTs with Multiple siRNAs.

Single-walled carbon nanotubes (SWCNTs) are produced using a high-pressure carbon monoxide (HiPco) process. The raw HiPco SWCNT product is added to an aqueous buffer solution (100 mM KCl, 30 mM HEPES-KOH [pH 7.5], 1 mM MgCl₂) containing a 20 μM mixture of two or more solubilized pooled siRNA [(siRNA targeting HIF-1α 5′-CCUGUGUCUAAAUCUGAAC-3′ (SEQ ID NO:6), 5′CUACCUUCGUGAUUCUGUUU-3′ (SEQ ID NO:7), GCACAAUAGACAGCGAAAC-3′ (SEQ ID NO:8), 5′-CUACUUUCUUAAUGGCUUA (SEQ ID NO:9), polo-like kinase 1 (PLK 1), 5′-CAACCAAAGUCGAAUAUUGAUU-3 (SEQ ID NO:10), 5′-C AAGAAGAAUGAAUACAGUUU-3′ (SEQ ID NO:11), 5′-GAAGAUGUCCAUGGAAAUAUU-3′ (SEQ ID NO:12), 5′-CAACACGCCUCAUCCUCUAUU-3′ (SEQ ID NO:13), Kinesin superfamily protein (Kif11), 5′-CGUCUUUAGAUUCCUAUAU-3′ (SEQ ID NO:14), 5′-GUUGUUCCUACUUCAGAUA-3′ (SEQ ID NO:15), 5′-GUCGUCUUUAGAUUCCU AU-3′ (SEQ ID NO:16), 5% GAUCUACCGAAAGAGUCAU-3′ (SEQ ID NO:17)], non-targeting siRNA 5′-UAGCGACAUUUGUGUAGUU-3′ (SEQ ID NO:18) and/or siTox, purchased from Dharmacon Inc., IL. This mixture is sonicated (Sonics, Vibra-cell) at 25° C. using two 15 second pulses at settings of 130 W, 20 k Hz, and 40% amplitude. The sonicated sample is centrifuged at 15,000×g for 5 minutes. The pellet comprising bundled SWCNTs is discarded and the supernatant is transferred into a clean tube and centrifuged an additional 1 minute at the same settings. The resulting supernatant contains SWCNTs noncovalently suspended by coatings of adsorbed siRNA. Near infrared (NIR) fluorescence spectroscopy may indicate that the sample contains predominantly individually suspended SWCNTs rather than nanotube aggregates.

### Example 1.2.

### Preparation of Noncovalent Complexes of SWCNTs with Multiple siRNA

SWCNTs are produced using a high-pressure carbon monoxide (HiPco) process. The raw HiPco SWCNT product is added to an aqueous buffer solution (100 mM KCl, 30 mM HEPES-KOH [pH 7.5], 1 mM MgCl₂) containing 20 μM of solubilized pooled single siRNA [(siRNA targeting HIF-1α 5′-CCUGUGUCUAAAUCUGAAC-3′ (SEQ ID NO:6), 5′CUACCUUCGUGAUUCUGUUU-3′ (SEQ ID NO:7), GCACAAUAGACAGCGAAAC-3′ (SEQ ID NO:8), 5′-CUACUUUCUUAAUGGCUUA (SEQ ID NO:9), polo-like kinase 1 (PLK1), 5′-CAACCAAAGUCGAAUAUUGAUU-3 (SEQ ID NO:10), 5′-CAAGAAGAAUGAAUACAGUUU-3′ (SEQ ID NO:11), 5′-GAAGAUGUCCAUGGAAAUAUU-3′ (SEQ ID NO:12), 5′-CAACACGCCUCAUCCUCUAUU-3′ (SEQ ID NO:13), Kinesin superfamily protein (Kif11), 5′-CGUCUUUAGAUUCCUAUAU-3′ (SEQ ID NO:14), 5′-GUUGUUCCUACUUCAGAUA-3′ (SEQ ID NO:15), 5′-GUCGUCUUUAGAUUCCU AU-3′ (SEQ ID NO:16), 5′-GAUCUACCGAAAGAGUCAU-3′ (SEQ ID NO:17)], non-targeting siRNA 5′-UAGCGACAUUUGUGUAGUU-3′ (SEQ ID NO:18) and/or siTox, purchased from Dharmacon Inc., IL. This mixture is sonicated (Sonics, Vibra-cell) at 25° C. using two 15 second pulses at settings of 130 W, 20 k Hz, and 40% amplitude. The sonicated sample is centrifuged at 15,000×g for 5 minutes. The pellet comprising bundled SWCNTs is discarded and the supernatant is transferred into a clean tube. To the SWCNT pellet is added an aqueous buffer solution (100 mM KCl, 30 mM HEPES-KOH [pH 7.5], 1 mM MgCl₂) containing 20 μM of a solubilized pooled second or third siRNA from the recited list above. This mixture is sonicated (Sonics, Vibra-cell) at 25° C. using two 15 second pulses at settings of 130 W, 20 k Hz, and 40% amplitude. The sonicated sample is centrifuged at 15,000×g for 5 minutes. The pellet comprising of any remaining bundled SWCNTs is discarded and the supernatant is transferred into a clean tube and is centrifuged an additional 1 minute at the same settings. The resulting supernatant may contain SWCNTs noncovalently suspended by coatings of adsorbed multiple siRNAs. Near infrared (NIR) fluorescence spectroscopy may indicate that the sample contains predominantly individually suspended SWCNTs rather than nanotube aggregates.

### Example 1.3

### Preparation of Noncovalent Complexes of SWCNTs with Multiple siRNA

SWCNTs were produced using a high-pressure carbon monoxide (HiPco) process. The raw HiPco SWCNT product was added to a 22.06 μM solution of 0.9% NaCl and solubilized pooled siRNA targeting different genes, siThioredoxin, and siEGFR [siRNA targeting Thioredoxin (TRX) 5′ CCAGUUGCCAUCUGCGUGA[dT][dT] 3′ (SEQ. ID. NO:21), 4 siRNAs targeting Epidermal growth factor receptor (EGFR) 5′ CUAUGUGCAGAGGAAUUAU[dT][dT] 3′ (SEQ. ID. NO:25), 5′ GAUCUUUCCUUCUUAAAGA[dT][dT] 3′ (SEQ. ID. NO:26), 5′ GAGGAAAUAUGUACUACGA[dT][dT] 3′ (SEQ. ID. NO:27), 5′ GACAUAGUCAGCAGUGACU[dT][dT] 3′ (SEQ. ID. NO:28)), purchased from Sigma Aldrich, St. Louis, Mo.]

This mixture was tip sonicated for a total of 2 minutes (Sonics, Vibra-cell) at 25° C. using 15-second pulses at settings of 130 W, 20 k Hz, and 40% amplitude. The sonicated sample was centrifuged at 17,800×g for 10 minutes. The pellet comprising bundled SWCNTs was discarded and the supernatant was transferred into a clean 1.7 mL microcentrifuge tube. The resulting supernatant contained SWCNTs noncovalently suspended by coatings of adsorbed multiple siRNAs. Near infrared (NIR) fluorescence spectroscopy indicated that the sample contained predominantly individually suspended SWCNTs rather than nanotube aggregates at a concentration of 39 μg/mL.

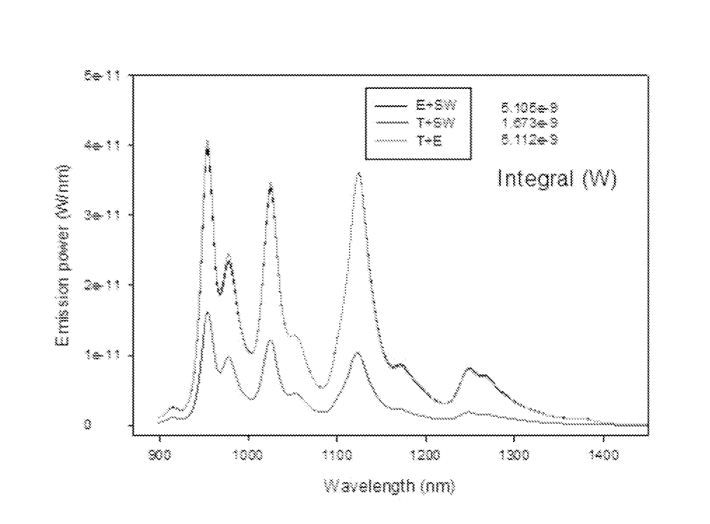

FIG. 2 shows the emission fluorescence spectrum of SWCNT solutions of siEGFR single payload (E+SW), siTRX single payload (T+SW) and siEGFR/siTRX SWCNT double payload. Spectrum shows that SWCNT are well dispersed in solution and The total fluorescence detected (659 nm excitation) is 5.11 nW siEGFR SWCNT, 1.67 nW siTRX SWCNT and 5.11 for siEGFR/siTrx SWCNT providing concentrations of 108 mg/L, 40 mg/L and 56 mg/L respectively.

Those of skill in the art will recognize that similar approaches may be made targeting different genes. For example, a similar procedure is contemplated for two siRNAs targeting v-Ki-ras2 Kirsten rat sarcoma 2 viral oncogene homolog (KRAS). The sense strands of those siRNAs comprise 5′ GUGCAAUGAGGGACCAGUA[dT][dT]3′(SEQ. ID. NO:29), or 5′ GUCUCUUGGAUAUUCUCGA[dT][dT] 3′ (SEQ. ID. NO:30), also purchased from Sigma Aldrich, St. Louis, Mo.

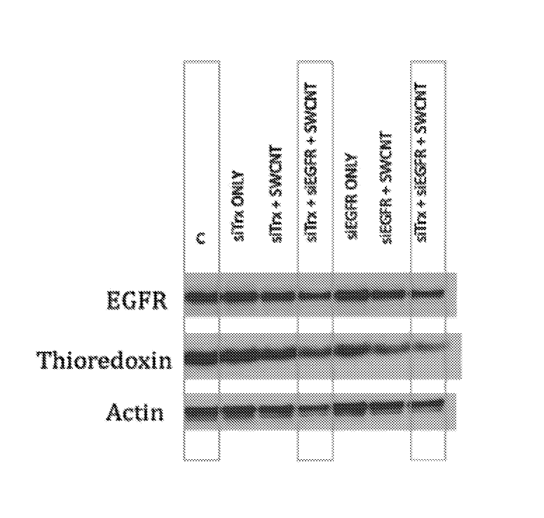

FIG. 1 depicts a Western blot analysis of some embodiments of the invention.: To evaluate biological activity MiaPaCa2 human pancreatic cancer cells in 96-well plates were exposed to 20 μl SWCNT preparations for 24 hrs, solution was replaced and fresh media added and the protein target was quantitated by Western blotting 72 hrs later. Treatment condition included Control (C; no treatment), PL-PEG solubiliezed SWCNT (SWCNT no siRNA control), siTRX−NO SWCNT, siTRX+SWCNT (single payload), siEGFR−NO SWCNT, siEGFR+SWCNT (single payload), siTRX+siEGFR+SWCNT (double payload). Cells lysates were prepared and 20μ of protein lysates were separated by SDS-PAGE. Membranes were probed with primary antibodies against TRX, EGFR or actin. Protein ratios versus actin were quantified. siRNA alone had no effect of the respective protein levels. siTRX/SWCNT single payload reduced Trx protein by 25%, siRNA EGFR single payload reduced EGFR protein by 20% resulting in an accompanying reduction in Trx by 50%. Effect of EGFR on Trx protein levels is a known associated event. siTrx/siEGFR/SWCNT double payload resulted in a KD of Trx by 28% and 63%, KD of EGFR by 16% and 33% in the two lane respectively.

### Analysis

To evaluate biological activity MiaPaCa2 human pancreatic cancer cells were exposed exposed to SWCNT preparations for 24 hrs and then the protein target was quantitated by Western blotting. Treatment condition included Control (C; no treatment), PL-PEG solubilized SWCNT (SWCNT no siRNA control), siTRX−NO SWCNT, siTRX+SWCNT (single payload), siEGFR−NO SWCNT, siEGFR+SWCNT (single payload), siTRX+siEGFR+SWCNT (double payload). 96-well plates were seeded with MiaPaCa2 human pancreatic cancer cells at a cell density of 5000 cells per well growing in 100 uL of DMEM media containing 10% fetal bovine calf serum. Cells were allowed to adhere for 24 hours before transfection with SWCNT solutions at 37° C./5%CO₂. Volumes of 10, 20, 40 or 80 μl of the SWCNT/siRNA solutions, siRNA alone in 0.9% NaCl, or SWCNT alone in a PL-PEG/0.9% NaCl solution were added to multiples of 3 wells After transfection, cells were incubated for 72 hours before harvesting cells for protein lysates. Transfection media was removed and cells were washed in 1×PBS buffered solution. After washing, wells containing adherent cells were treated with 100 uL of cell lysis buffer and protease inhibitor. Three wells of each treatment condition were removed from wells and placed in microcentrifuge tubes. Cells and lysis buffer with protease inhibitor were vortexed every 10 minutes for a total of 30 minutes and then centrifuged at 17,800×g for 30 minutes at 4 degrees Celsius. Cell lysate supernatant was removed from cell pellet and transferred to fresh 1.7 mL microcentrifuge tube and stored in −20 degrees Celsius conditions for future use.

Twenty microliters of protein lysates were separated by SDS-PAGE using 12% Criterion XT Bis-Tris polyacrylamide gel at 110V for 1.5 hours and transferred to a PVDF membrane. Proteins were transferred from polyacrylamide gel to PVDF membrane at 20V for 18 hours. After protein separation and transfer to PVDF membrane, membrane was blocked using 5% NFDM and TBS-T solution. Membranes were probed separately with primary antibodies against Thioredoxin (sc-20146) and EGFR (sc-71034) all purchased from Santa Cruz Biotechnology (Santa Cruz, Calif.) and actin for control. Membrane was washed between primary and secondary antibody incubation with TBS-T buffer solution. Antibody-antigen complexes were visualized with HRP conjugated-secondary antibodies and HRP chemiluminescent detection system purchased from Perkin Elmer (Waltham, Mass.).

In a study using both siTrx and siEGFR payloads on SWCNT, it was found that the siRNA alone and the SWCNT alone had no effect on the cells' growth or specific protein level. Single payload siEGFR or siTRX each produced the respective protein knockdown (KD) in a dose dependent manner and siEGFR KD resulted cell killing in a dose depended manner as expected (results not shown). Using SWCNT/double siRNA payload with siTrx and siEGFR it was found that the growth factor EGFR and siTRX payloads diminished respective protein levels as illustrated in FIG. 1 for those cells exposed to 20 μl of the solutions prepared as described below.

FIG. 1 depicts a Western blot analysis of some embodiments of the invention. To evaluate biological activity MiaPaCa2 human pancreatic cancer cells in 96-well plates were exposed to 20 μl SWCNT preparations for 24 hrs, solution was replaced and fresh media added and the protein target was quantitated by Western blotting 72 hrs later. Treatment condition included Control (C; no treatment), PL-PEG solubiliezed SWCNT (SWCNT no siRNA control), siTRX−NO SWCNT, siTRX+SWCNT (single payload), siEGFR−NO SWCNT, siEGFR+SWCNT (single payload), siTRX+siEGFR+SWCNT (double payload). Cells lysates were prepared and 20μ of protein lysates were separated by SDS-PAGE. Membranes were probed with primary antibodies against TRX, EGFR or actin. Protein ratios versus actin were quantified. siRNA alone had no effect of the respective protein levels. siTRX/SWCNT single payload reduced Trx protein by 25%, siRNA EGFR single payload reduced EGFR protein by 20% resulting in an accompanying reduction in Trx by 50%. Effect of EGFR on Trx protein levels is a known associated event. siTrx/siEGFR/SWCNT double payload resulted in a KD of Trx by 28% and 63%, KD of EGFR by 16% and 33% in the two lane respectively.

### Preparation of siRNA Stock Solutions

preparation of siEGFR stock solution 1, 9.9 nmoles siRNA targeting Epidermal Growth Factor Receptor (EGFR) (Sigma Aldrich) was dissolved in nuclease-free water (Ambion) to a final volume of 1 mL. EGFR siRNA sequence is 5′ CUAUGUGCAGAGGAAUUAU[a][dT] 3′.

For preparation of siEGFR stock solution 2, 9.8 nmoles siRNA targeting Epidermal Growth Factor Receptor (EGFR) (Sigma Aldrich) was dissolved in nuclease-free water (Ambion) to a final volume of 1 mL. EGFR siRNA sequence is 5′ GAUCUUUCCUUCUUAAAGA[dT][dT] 3′.

For preparation of siEGFR stock solution 3, 10.0 nmoles siRNA targeting Epidermal Growth Factor Receptor (EGFR) (Sigma Aldrich) was dissolved in nuclease-free water (Ambion) to a final volume of 1 mL. EGFR siRNA sequence is 5′ GAGGAAAUAUGUACUACGA[dT][dT] 3′

For preparation of siEGFR stock solution 4, 10.2 nmoles siRNA targeting Epidermal Growth Factor Receptor (EGFR) (Sigma Aldrich) was dissolved in nuclease-free water (Ambion) to a final volume of 1 mL. EGFR siRNA sequence is 5′GACAUAGUCAGCAGUGACU[dT][dTY] 3′.

For preparation of siTRX stock solutions, 258.1 nmoles siRNA targeting Thioredoxin (Sigma Aldrich) was dissolved in nuclease-free water (Ambion) to a final volume of 1 mL. Thioredoxin siRNA sequence is 5′ CCAGUUGCCAUCUGCGUGA[dT][dT] 3′.

The concentration of siRNA was determined by measuring UV absorbance at 260 nm and 280 nm using NanoDrop™ 1000 (Thermo Fisher Scientific).

### Preparation of siTrx—No SWCNT Solution

siTRX stock solution targeting Thioredoxin (TRX) was added to 0.9% NaCl to make a final solution of 0.1469 μg/μL.

### Preparation of siEGFR—No SWCNT Solution

Four different siEGFR stock solutions 1 to 4 targeting Epidermal Growth Factor Receptor (EGFR) were combined with 0.9% NaCl to make a final siEGFR solution of 0.1469 μg/μL.

### Preparation OF siEGFR/siTRX Solution

Equal volumes of four different siEGFR stock solutions 1 to 4 targeting Epidermal Growth Factor:Receptor (EGFR) were combined with 0.9% NaCl to make a final siEGFR solution of 0.2938 μg/μL. siTRX stock solution targeting Thioredoxin (TRX) was added to 0.9% NaCl to make a final solution of 0.2938 μg/μL. Equal volumes of each siRNA solution were combined to make a final double siEGFR/siTRX solution at 0.2938 μg/μL.

### Double Payload SWCNT Preparation (siEGFR and siTRX Double Payload)

Raw HiPco SWCNTs (Lot HPR 188.4) (SWCNT) were dispersed in 0.2938 μg/μL siEGFR/siTrx/0.9% NaCl solution. 110 μg dry SWCNT were added to a 680.62 μL of this siEGFR/siTrx/NaCl solution. The mixture was tip sonicated for 2 minutes (Sonics, Vibra-cell) at 25° C. using 15 second pulses at settings of 130 W, 20 k Hz, and 40% amplitude. Between 15 second periods of sonication, sample was placed in ice for 45 seconds. The sonicated sample was centrifuged at 17,800×g for 10 minutes. Supernatant was removed and transferred to 1.7 mL microcentrifuge tube.

### Single Payload SWCNT Preparation (siEGFR or siTrx Single Payloads)

Raw HiPco SWCNTs (Lot HPR 188.4) 110 μg were dispersed in 680.62 μL siEGFR in 0.9% NaCl (0.1469 μg/μL) or siTRX in 0.9% NaCl (0.1469 μg/μL). The mixtures were tip sonicated for 2 minutes (Sonics, Vibra-cell) at 25° C. using 15 second pulses at settings of 130 W, 20 k Hz, and 40% amplitude. Between 15 second periods of sonication, sample was placed in ice. for 45 seconds. The sonicated samples were centrifuged at 17,800×g for 10 minutes. Supernatants were removed and transferred to 1.7 mL microcentrifuge tube.

### Preparation of PL-PEG/SWCNT Solution

For preparation of PL-PEG solution, 8.4 μL of 5.952×10⁻³ M PL-PEG stock (10 mg/mL) was added to 491 μL of 0.9%NaCl for a final volume of 500 μL. This solution was added to 110 μg SWCNT. The mixture was tip sonicated for 2 minutes (Sonics, Vibra-cell) at 25° C. using 15 second pulses at settings of 130 W, 20 k Hz, and 40% amplitude. Between 15 second periods of sonication, sample was placed in ice for 45 seconds. The sonicated sample was centrifuged at 17,800×g for 10 minutes. Supernatant was removed and transferred to 1.7 mL microcentrifuge tube.

### Preparation of PL-PEG Stock Solution

For preparation PL-PEG stock solution, powdered PL-PEG purchased from Avanti Polar Lipids (Alabaster, Ala., USA) (14:0 PEG5000 PE (1,2-dimyristoyl-sn-glycero-3-phosphoethanolamine-N-[methoxy(polyethylene glycol)-5000] (ammonium salt)) was solubilized in 2.5 mL dimethyl sulfoxide (Sigma) for a final molar concentration of 5.952×10⁻³ (10 mg/mL).

Various modifications of the invention, in addition to those described herein, will be apparent to those skilled in the art from the foregoing description. Such modifications are intended to fall within the scope of the appended claims.

1. A pharmaceutical composition comprising:
a fullerene carbon nanotube;
a first siRNA complexed with the fullerene carbon nanotube;
at least a second siRNA complexed with the fullerene carbon nanotube; and
a pharmaceutically acceptable carrier, wherein the first siRNA is targeted, and
the second siRNA is selected from an untargeted siRNA or a targeted siRNA.

2. The pharmaceutical composition: of claim 1, wherein
the second siRNA noncovalently solubilizes the fullerene carbon nanotube into the pharmaceutically acceptable carrier.

3. The pharmaceutical composition of claim 1,
wherein the first siRNA is targeted to a first target and the second siRNA is targeted to a second target.

4. The pharmaceutical composition of claim 1, further comprising at least a third siRNA complexed with the fullerene carbon nanotube.

5. The pharmaceutical composition of claim 1, wherein the fullerene carbon nanotube is unagglomerated and nonaggregated.

6. The pharmaceutical composition of claim 1, wherein the diameter of the fullerene carbon nanotube is about 1-5 nm.

7. The pharmaceutical composition of claim 1, wherein the diameter of the fullerene carbon nanotube is about 1 nm.

8. The pharmaceutical composition of claim 1, wherein the length of the fullerene carbon nanotube is about 500 nm or less.

9-13. (canceled)

14. The pharmaceutical composition of claim 1, wherein the first siRNA comprises an siRNA selected from a chemically-modified siRNA, or a stabilized siRNA.

15. (canceled)

16. The pharmaceutical composition of claim 1, wherein the second siRNA comprises stabilized siRNA.

17. The pharmaceutical composition of claim 1, wherein the first siRNA is targeted to a target selected from vascular endothelial growth factor (VEGF) mRNA, endothelial growth factor receptor (EGFR) mRNA, human epidermal growth factor receptor 2 (HER2) mRNA; hypoxia-inducible factor 1 alpha (HIF-1α) mRNA, polo-like kinase 1 (PLK1); Kinesin superfamily protein (Kif11), Thioredoxin (TRX) mRNA, and v-Ki-ras2 Kirsten rat sarcoma 2 viral oncogene homolog (KRAS) mRNA.

18. The pharmaceutical composition of claim 16, wherein the first siRNA is targeted to vascular endothelial growth factor (VEGF) mRNA and wherein the sense strand of the first siRNA is AUGUGAAUGCAGACCAAAGAA (SEQ ID NO: 1).

19. (canceled)

20. The pharmaceutical composition of claim 1, wherein the first siRNA is targeted to endothelial growth factor receptor (EGFR) mRNA and wherein the sense strand of the first siRNA comprises a sequence selected from GUCAGCCUGAACAUAACAU (SEQ ID NO: 2), and GUGUAACGGAAUAGGUAUU (SEQ ID NO: 3).

21. (canceled)

22. The pharmaceutical composition of claim 1, wherein the first siRNA is targeted to human epidermal growth factor receptor 2 (HER2) mRNA and wherein the sense strand of the first siRNA comprises a sequence selected from GGAGCUGGCGGCCUUGUGCCG (SEQ ID NO: 4), and UCACAGGGGCCUCCCCAGGAG (SEQ ID NO: 5).

23. (canceled)

24. The pharmaceutical composition of claim 1, wherein the first siRNA is targeted to hypoxia-inducible factor 1 alpha (HIF-1α) mRNA and wherein the sense strand of the first siRNA comprises a sequence selected from CCUGUGUCUAAAUCUGAAC (SEQ ID NO:6), CUACCUUCGUGAUUCUGUUU (SEQ ID NO:7), GCACAAUAGACAGCGAAAC (SEQ ID NO:8), CUACUUUCUUAAUGGCUUA (SEQ ID NO:9), 5′ CAAAUACAUGGGAUUAACU[dT][dT]3′ (SEQ. ID. NO:19) and 5′ GCAACUUGAGGAAGUACCA[dT][dT]3′ (SEQ. ID. NO: 20).

25. (canceled)

26. The pharmaceutical composition of claim 1, wherein the first siRNA is targeted to polo-like kinase 1 (PLK1) and wherein the sense strand of the first siRNA comprises a sequence selected from CAACCAAAGUCGAAUAUUGAUU (SEQ ID NO:10), CAAGAAGAAUGAAUACAGUUU (SEQ ID NO:11), GAAGAUGUCCAUGGAAAUAUU (SEQ ID NO:12), and AACACGCCUCAUCCUCUAUU (SEQ ID NO:13).

27. (canceled)

28. The pharmaceutical composition of claim 1, wherein the first siRNA is targeted to Kinesin superfamily protein (Kif11) and wherein the sense strand of the first siRNA comprises a sequence selected from CGUCUUUAGAUUCCUAUAU (SEQ ID NO:14), GUUGUUCCUACUUCAGAUA (SEQ ID NO:15), GUCGUCUUUAGAUUCCUAU (SEQ ID NO:16), and GAUCUACCGAAAGAGUCAU (SEQ ID NO:17).

29. (canceled)

30. The pharmaceutical composition of claim 1, wherein the first siRNA is targeted to Thioredoxin (TRX) mRNA and wherein the sense strand of the first siRNA comprises a sequence selected from CCAGUUGCCAUCUGCGUGA (SEQ. ID NO: 21), 5′ CUUGGACGCUGCAGGUGAU[dT][dT] 3′ (SEQ.ID.NO:22), 5′ AUUCCAACGUGAUAUUCCU[dT][dT] 3′ (SEQ.ID.NO:23), and 5′ GCCAUCUGCGUGACAAUAA[dT][dT] 3′ (SEQ.ID.NO:24).

31. (canceled)

32. The pharmaceutical composition of claim 1, wherein the first siRNA is targeted to Epidermal growth factor receptor (EGFR) mRNA and wherein the sense strand of the first siRNA comprises a sequence selected from CUAUGUGCAGAGGAAUUAU (SEQ. ID. NO:25), GAUCUUUCCUUCUUAAAGA (SEQ. ID. NO:26), GAGGAAAUAUGUACUACGA (SEQ. ID. NO:27), and GACAUAGUCAGCAGUGACU (SEQ. ID. NO:28).

33. (canceled)

34. The pharmaceutical composition of claim 1, wherein the first siRNA is targeted to v-Ki-ras2 Kirsten rat sarcoma 2 viral oncogene homolog (KRAS) mRNA and wherein the sense strand of the first siRNA comprises a sequence selected from GUGCAAUGAGGGACCAGUA (SEQ. ID. NO:29), and GUCUCUUGGAUAUUCUCGA (SEQ. ID. NO:30).

35. The pharmaceutical composition of claim 1, wherein the second siRNA is targeted to a target selected from vascular endothelial growth factor (VEGF) mRNA, endothelial growth factor receptor (EGFR) mRNA, human epidermal growth factor receptor 2 (HER2) mRNA; hypoxia-inducible factor 1 alpha (HIF-1α) mRNA, polo-like kinase 1 (PLK1); Kinesin superfamily protein (Kif11), Thioredoxin (TRX) mRNA, and v-Ki-ras2 Kirsten rat sarcoma 2 viral oncogene homolog (KRAS) mRNA.

36. The pharmaceutical composition of claim 35, wherein the second siRNA is targeted to vascular endothelial growth factor (VEGF) mRNA and wherein the sense strand of the second siRNA is AUGUGAAUGCAGACCAAAGAA (SEQ ID NO: 1).

37. (canceled)

38. The pharmaceutical composition of claim 37, wherein the second siRNA is targeted to endothelial growth factor receptor (EGFR) mRNA and wherein the sense strand of the second siRNA comprises a sequence selected from GUCAGCCUGAACAUAACAU (SEQ ID NO: 2), and GUGUAACGGAAUAGGUAUU (SEQ ID NO: 3).

39. (canceled)

40. The pharmaceutical composition of claim 1, wherein the second siRNA is targeted to human epidermal growth factor receptor 2 (HER2) mRNA and wherein the sense strand of the second siRNA comprises a sequence selected from GGAGCUGGCGGCCUUGUGCCG (SEQ ID NO: 4), and UCACAGGGGCCUCCCCAGGAG (SEQ ID NO: 5).

41. (canceled)

42. The pharmaceutical composition of claim 1, wherein the second siRNA is targeted to hypoxia-inducible factor 1 alpha (HIF-1α) mRNA and wherein the sense strand of the second siRNA comprises a sequence selected from CCUGUGUCUAAAUCUGAAC (SEQ ID NO:6), CUACCUUCGUGAUUCUGUUU (SEQ ID NO:7), GCACAAUAGACAGCGAAAC (SEQ ID NO:8), and CUACUUUCUUAAUGGCUUA (SEQ ID NO:9).

43. (canceled)

44. The pharmaceutical composition of claim 1, wherein the second siRNA is targeted to polo-like kinase 1 (PLK1) and wherein the sense strand of the second siRNA comprises a sequence selected from CAACCAAAGUCGAAUAUUGAUU (SEQ ID NO:10), CAAGAAGAAUGAAUACAGUUU (SEQ ID NO:11), GAAGAUGUCCAUGGAAAUAUU (SEQ ID NO:12), and CAACACGCCUCAUCCUCUAUU (SEQ ID NO:13).

45. (canceled)

46. The pharmaceutical composition of claim 1, wherein the second siRNA is targeted to Kinesin superfamily protein (Kif11 ) and wherein the sense strand of the second siRNA comprises a sequence selected from CGUCUUUAGAUUCCUAUAU (SEQ ID NO:14), GUUGUUCCUACUUCAGAUA (SEQ ID NO:15), GUCGUCUUUAGAUUCCUAU (SEQ ID NO:16), and GAUCUACCGAAAGAGUCAU (SEQ ID NO:17).

47. (canceled)

48. The pharmaceutical composition of claim 1, wherein the second siRNA is targeted to Thioredoxin (TRX) mRNA and wherein the sense strand of the second siRNA comprises a sequence selected from CCAGUUGCCAUCUGCGUGA (SEQ. ID NO: 21), 5′ CUUGGACGCUGCAGGUGAU[dT][dT] 3′ (SEQ.ID.NO:22), 5′ AUUCCAACGUGAUAUUCCU[dT][dT] 3′ (SEQ.ID.NO:23), and 5′ GCCAUCUGCGUGACAAUAA[dT][dT] 3′ (SEQ.ID.NO:24).

49. (canceled)

50. The pharmaceutical composition of claim 1, wherein the second siRNA is targeted to Epidermal growth factor receptor (EGFR) mRNA and wherein the sense strand of the second siRNA comprises a sequence selected from CUAUGUGCAGAGGAAUUAU (SEQ. ID. NO:25), GAUCUUUCCUUCUUAAAGA (SEQ. ID. NO:26), GAGGAAAUAUGUACUACGA (SEQ. ID. NO:27), and GACAUAGUCAGCAGUGACU (SEQ. ID. NO:28).

51. (canceled)

52. The pharmaceutical composition of claim 1, wherein the second siRNA is targeted to v-Ki-ras2 Kirsten rat sarcoma 2 viral oncogene homolog (KRAS) mRNA and wherein the sense strand of the second siRNA comprises a sequence selected from GUGCAAUGAGGGACCAGUA (SEQ. ID. NO:29), and GUCUCUUGGAUAUUCUCGA (SEQ. ID. NO:30)).

53. The pharmaceutical composition of claim 1, wherein pharmaceutically acceptable carrier is liquid.

54. The pharmaceutical composition of claim 1, wherein pharmaceutically acceptable carrier is selected from water, an isotonic salt solution, an isotonic sugar solution, is an aqueous polyethylene glycol (PEG) solution, an organic solvent dissolved in isotonic aqueous solution, and an aqueous buffer solution.

55-59. (canceled)

60. The pharmaceutical composition of claim 1, further comprising a functional group, wherein such functional group links the first siRNA and/or at least the second siRNA with the fullerene carbon nanotube.

61. The pharmaceutical composition of claim 60, wherein the functional group is polyethylene glycol (PEG).

62. The pharmaceutical composition of claim 1, further comprising one or more bioactive agents.

63. The pharmaceutical composition of claim 1, wherein said pharmaceutical composition provides delivery of an effective amount of the first siRNA, and wherein said effective amount reduces the expression of a target nucleic acid when compared to siRNA not complexed to the fullerene carbon nanotube.

64. The pharmaceutical composition of claim 1, wherein said pharmaceutical composition provides delivery of an effective amount of said at least a second siRNA, and wherein said effective amount reduces the expression of a target nucleic acid when compared to siRNA not complexed to the fullerene carbon nanotube.

65. A fullerene carbon nanotube composition comprising a fullerene carbon nanotube, a first bioactive agent complexed with the fullerene carbon nanotube, at least a second bioactive agent complexed with the fullerene carbon nanotube, and a pharmaceutically acceptable carrier, wherein the fullerene carbon nanotube composition is internalized in treated cells in media containing serum at a rate measured in vitro that substantially corresponds to the following:
(i) from about 0.01 to about 30% of the total amount of treated cells internalize the fullerene carbon nanotube composition after about 1 hour of measurement;
(ii) from about 20 to about 90% of the total amount of treated cells internalize the fullerene carbon nanotube composition after about 3 hours of measurement; and
(iii) not less than about 95% of the total amount of treated cells internalize the fullerene carbon nanotube composition after about 24 hours of measurement.

66. A method of reducing the expression of a targeted gene in cell culture, comprising delivering an effective amount of a fullerene carbon nanotube composition comprising a fullerene carbon nanotube, a first siRNA complexed with the fullerene carbon nanotube, at least a second siRNA complexed with the fullerene carbon nanotube, and a pharmaceutically acceptable carrier, wherein the first siRNA is targeted and the second siRNA is untargeted.

67. A method of reducing the expression of a targeted gene in cell culture, comprising delivering an effective amount of a fullerene carbon nanotube composition comprising a fullerene carbon nanotube, a first siRNA complexed with the fullerene carbon nanotube, at least a second siRNA complexed with the fullerene carbon nanotube, and a pharmaceutically acceptable carrier, wherein the first siRNA is targeted and the second siRNA noncovalently solubilizes the fullerene carbon nanotube into the pharmaceutically acceptable carrier.

68. A method of reducing the expression of a targeted gene in cell culture, comprising delivering an effective amount of a fullerene carbon nanotube composition comprising a fullerene carbon nanotube, a first siRNA complexed with the fullerene carbon nanotube, at least a second siRNA complexed with the fullerene carbon nanotube, and a pharmaceutically acceptable carrier, wherein the first siRNA is targeted to a first target and the second siRNA is targeted to a second target.

69. A method of effectively silencing a targeted gene in vivo, comprising administering to a subject an effective amount of a fullerene carbon nanotube composition comprising a fullerene carbon nanotube, a first siRNA complexed with the fullerene carbon nanotube, at least a second siRNA complexed with the fullerene carbon nanotube, and a pharmaceutically acceptable carrier, wherein the first siRNA is targeted and the second siRNA is untargeted.

70. A method of effectively silencing a targeted gene in vivo, comprising administering to a subject an effective amount of a fullerene carbon nanotube composition comprising a fullerene carbon nanotube, a first siRNA complexed with the fullerene carbon nanotube, at least a second siRNA complexed with the fullerene carbon nanotube, and a pharmaceutically acceptable carrier, wherein the first siRNA is targeted and the second siRNA noncovalently solubilizes the fullerene carbon nanotube into the pharmaceutically acceptable carrier.

71. A method of effectively silencing a targeted gene in vivo, comprising administering to a subject an effective amount of a fullerene carbon nanotube composition comprising a fullerene carbon nanotube, a first siRNA complexed with the fullerene carbon nanotube, at least a second siRNA complexed with the fullerene carbon nanotube, and a pharmaceutically acceptable carrier, wherein the first siRNA is targeted to a first target and the second siRNA is targeted to a second target.


SEQUENCE LISTING (30 sequences)

SEQ ID NO: 1 - RNA, 21 nt, Artificial Sequence
  VEGF siRNA
augugaaugcagaccaaagaa

SEQ ID NO: 2 - RNA, 19 nt, Artificial Sequence
  EGFR siRNA
gucagccugaacauaacau

SEQ ID NO: 3 - RNA, 19 nt, Artificial Sequence
  EGFR siRNA 2
guguaacggaauagguauu

SEQ ID NO: 4 - RNA, 21 nt, Artificial Sequence
  HER2 siRNA
ggagcuggcggccuugugccg

SEQ ID NO: 5 - RNA, 21 nt, Artificial Sequence
  HER2 siRNA 2
ucacaggggccuccccaggag

SEQ ID NO: 6 - RNA, 19 nt, Artificial Sequence
  HIF-1a siRNA
ccugugucuaaaucugaac

SEQ ID NO: 7 - RNA, 20 nt, Artificial Sequence
  HIF-1a si RNA 2
cuaccuucgugauucuguuu

SEQ ID NO: 8 - RNA, 19 nt, Artificial Sequence
  HIF1a siRNA 3
gcacaauagacagcgaaac

SEQ ID NO: 9 - RNA, 19 nt, Artificial Sequence
  HIF1a siRNA 4
cuacuuucuuaauggcuua

SEQ ID NO: 10 - RNA, 22 nt, Artificial Sequence
  PLK1 siRNA 1
caaccaaagucgaauauugauu

SEQ ID NO: 11 - RNA, 21 nt, Artificial Sequence
  PLK1 siRNA 2
caagaagaaugaauacaguuu

SEQ ID NO: 12 - RNA, 21 nt, Artificial Sequence
  PLK1 siRNA 3
gaagauguccauggaaauauu

SEQ ID NO: 13 - RNA, 21 nt, Artificial Sequence
  PLK1 siRNA 4
caacacgccucauccucuauu

SEQ ID NO: 14 - RNA, 19 nt, Artificial Sequence
  Kif11 siRNA 1
cgucuuuagauuccuauau

SEQ ID NO: 15 - RNA, 19 nt, Artificial Sequence
  Kif11 siRNA 2
guuguuccuacuucagaua

SEQ ID NO: 16 - RNA, 19 nt, Artificial Sequence
  Kif11 siRNA 3
gucgucuuuagauuccuau

SEQ ID NO: 17 - RNA, 19 nt, Artificial Sequence
  Kif11 siRNA 4
gaucuaccgaaagagucau

SEQ ID NO: 18 - RNA, 19 nt, Artificial Sequence
  non-targeting siRNA
uagcgacauuuguguaguu

SEQ ID NO: 19 - DNA, 21 bp, Artificial Sequence
  HIF1a hybrid siRNA 1
caaauacaugggauuaacunn

SEQ ID NO: 20 - DNA, 21 bp, Artificial Sequence
  HIF1a hybrid siRNA 2
gcaacuugaggaaguaccann

SEQ ID NO: 21 - DNA, 21 bp, Artificial Sequence
  TRX siRNA 1
ccaguugccaucugcgugann

SEQ ID NO: 22 - DNA, 21 bp, Artificial Sequence
  TRX siRNA 2
cuuggacgcugcaggugaunn

SEQ ID NO: 23 - DNA, 21 bp, Artificial Sequence
  TRX siRNA 3
auuccaacgugauauuccunn

SEQ ID NO: 24 - DNA, 21 bp, Artificial Sequence
  TRX siRNA 4
gccaucugcgugacaauaann

SEQ ID NO: 25 - DNA, 21 bp, Artificial Sequence
  EGFR siRNA 1
cuaugugcagaggaauuaunn

SEQ ID NO: 26 - DNA, 21 bp, Artificial Sequence
  EGFR siRNA 2
gaucuuuccuucuuaaagann

SEQ ID NO: 27 - DNA, 21 bp, Artificial Sequence
  EGFR siRNA 3
gaggaaauauguacuacgann

SEQ ID NO: 28 - DNA, 21 bp, Artificial Sequence
  EGFR siRNA 4
gacauagucagcagugacunn

SEQ ID NO: 29 - DNA, 21 bp, Artificial Sequence
  KRAS siRNA 1
gugcaaugagggaccaguann

SEQ ID NO: 30 - DNA, 21 bp, Artificial Sequence
  KRAS siRNA 2
gucucuuggauauucucgann